(19) United States
(12) Patent Application Publication
(10) Pub. No.: US 2025/0352072 A1
(43) Pub. Date: Nov. 20, 2025

(54) SYSTEMS AND METHODS FOR ASSESSING EJECTION FRACTION, HEART FAILURE, AND SLEEP APNEA USING ELECTROCARDIOGRAPHIC SIGNALS

(71) Applicant: Peerbridge Health, Inc., New York, NY (US)

(72) Inventor: Sandeep Gulati, New York, NY (US)

(21) Appl. No.: 19/209,521

(22) Filed: May 15, 2025

Related U.S. Application Data
(60) Provisional application No. 63/647,902, filed on May 15, 2024.

(51) Int. Cl. A61B 5/02; A61B 5/00; A61B 5/024; A61B 5/256; A61B 5/282; A61B 5/283; A61B 5/352; A61B 5/353; A61B 5/358; A61B 5/36; G16H 15/00; G16H 50/20; G16H 50/30

(52) U.S. Cl. CPC A61B 5/02028; A61B 5/02405; A61B 5/256; A61B 5/282; A61B 5/283; A61B 5/352; A61B 5/353; A61B 5/358; A61B 5/36; A61B 5/4818; A61B 5/6823; A61B 5/6824; A61B 5/6826; A61B 5/6852; A61B 5/6898; A61B 5/7207; G16H 15/00; G16H 50/20; G16H 50/30; A61B 2560/0468

(57) ABSTRACT

Described herein are systems and methods for characterizing a subject's ejection fraction (EF) status, heart failure status, and/or sleep apnea status. In particular, described herein are systems and methods for characterizing a subject's EF status, heart failure status, and/or sleep apnea status through use of a wearable electrocardiogram (ECG) device having LII and LIII leads, and software configured to incorporate data from the ECG device and assess the subject's EF status, heart failure status, and/or sleep apnea status.

### CROSS-REFERENCE TO RELATED APPLICATION

The present application claims priority to U.S. Provisional Application No. 63/647,902, filed May 15, 2024, which is incorporated herein by reference in its entirety.

### FIELD OF THE INVENTION

Described herein are systems and methods for characterizing a subject's ejection fraction (EF) status, heart failure status, and/or sleep apnea status. In particular, described herein are systems and methods for characterizing a subject's EF status, heart failure status, and/or sleep apnea status through use of a wearable electrocardiogram (ECG) device having LII and LIII leads, and software configured to incorporate data from the ECG device and assess the subject's EF status, heart failure status, and/or sleep apnea status.

### BACKGROUND

Heart failure (HF or congestive heart failure (CHF)) is a major public health concern with high morbidity and mortality and is the leading cause of hospitalization in older demographics (75+) in the US. Approximately half of affected patients are HF with reduced ejection fraction (EF) (HFrEF) and the other half with preserved EF (HFpEF). Worldwide the burden has increased to an estimated 64 million people. HFrEF is associated with severe impairment of physical and mental health status and carries a high 5-year mortality rate of ˜75%. Many individuals are asymptomatic, undetected, and untreated (see, e.g., Murtagh G, et al., Eur J Heart Fail 2012; 14:480-486).

The treatment of cardiovascular disease continues to improve, and important strides are made every year in extending the lives of people impacted by these conditions. However, despite these advancements, many patients are still plagued by a lack of accessibility to early diagnosis and affordable care. Direct-from-ECF EF using ambulatory electrocardiogram (ECG) wearables solves access and affordability by combining the power of artificial intelligence with remote ambulatory electrocardiograma screen, diagnose, and monitor patients with cardiovascular disease.

Left ventricular ejection fraction (LVEF) is a fundamental aspect of cardiac structure and function. Defined as the amount of blood the left ventricle pumps out to the body with each heartbeat, the EF and EF Severity can be impacted by a myriad of cardiovascular conditions, from coronary artery disease and myocardial infarction to valvular heart disease. Cardiomyopathy, or primary heart muscle disease, is most commonly associated with impaired LVEF and can be inherited or caused by hypertension, alcohol abuse, toxicity from chemotherapy, viral infection, or other causes. Impairment of cardiac pumping as evidenced by depressed LVEF is the most common hallmark of heart failure (HF), which impacts 5-6 million Americans and carries substantial morbidity and mortality.

Analysis of EF and EF Severity can be used to screen for, diagnose, and monitor response to therapy or progression of numerous cardiovascular diseases. Current modalities used for evaluating left ventricular ejection fraction include echocardiography (2D, 3D, and transesophageal), radionuclide ventriculography, invasive left ventriculography, and CT- or MRI-ventriculography. Each of these modalities requires patients to travel to a healthcare facility, requires specialized equipment and highly trained technicians to perform the studies, and is very expensive. Echocardiography is the most commonly used test to evaluate and monitor LVEF, but while it is relatively quick and very safe, it is not always easily accessible to patients in rural and underserved areas and is too expensive to be utilized for screening or for serial examinations over a short period of time.

Improved methods for assessing a subject's EF status are needed.

The present invention addresses these needs.

### SUMMARY

Experiments conducted during the course of developing embodiments for the present invention demonstrated 1) direct-from-ECG screening for heart failure with reduced and preserved ejection fraction: 2) a direct-from-ECG Apnea-Hypopnea Index (AHI) for improving AFib and obstructive sleep apnea; and 3) direct-from-ECG Apnea-Hypopnea Index (AHI) for improving AFib and obstructive sleep apnea.

Accordingly, the present invention provides systems and methods for characterizing a subject's ejection fraction (EF) status, heart failure status, and/or sleep apnea status. In particular, described herein are systems and methods for characterizing a subject's EF status, heart failure status, and/or sleep apnea status through use of a wearable electrocardiogram (ECG) device having LII and LIII leads, and software configured to incorporate data from the ECG device and assess the subject's EF status, heart failure status, and/or sleep apnea status.

In certain embodiments, the present invention provides a system for characterizing a subject's Ejection Fraction (EF) percentage status, comprising:
providing an electrocardiogram (ECG) device and a processor:
   wherein the ECG device is wearable, wherein the ECG device has LI and LIII leads, wherein the ECG device is configured upon placement onto a subject to capture LII and LIII electrical signal waveform data from the subject via the LII and LIII leads over an extended period of time, wherein the ECG device is configured to wirelessly transmit LII and LIII electrical signal waveform data from the subject captured over an extended period of time to a processor, and
   wherein the processor comprises software that, when executed, causes the processor to manually or automatically:
      receive LII and LIII electrical signal waveform data captured over an extended period of time from a subject wirelessly transmitted from the ECG device; and
      utilize the received LII and LIII electrical signal waveform data captured over an extended period of time to:
         assess the subject's ejection fraction percentage status; and
         provide a report to a user of the assessed ejection fraction percentage for the subject.

In some embodiments, the software, when executed, is configured to accomplish one or more of the following:
resample the LII and LIII electrical signal waveform data captured over an extended period of time to 250 Hz;
derive LI waveform with resampled LI and LII waveform data;
utilize the derived LI waveform and resampled LII and LIII waveform data to derive the following other bipolar frontal leads: aVF, aVR and aVL;
estimate a Wilson Central Terminal Reference potential with a spatial vector cardiography transform;
approximate a beat-by-beat maxima for pre-cordial leads V1 thru V6 with the spatial vector cardiography transform;
filter the derived LI waveform data, LII waveform data, LIII waveform data, aVF data, aVR data, aVL data, Wilson Central Terminal Reference potential, and V1 thru V6 data to remove motion (and other) artifacts;
calculate a spectral entropy for each heartbeat over the time period and remove all heartbeats above a pre-set threshold model;
compute PR, QT, ST interval for remaining stable heart-beats;
compute windowed HRV;
measure ventricular activation time (VAT) for derived lead aVR, wherein the VAT is measured from the onset of the QRS complex to the peak of the R wave;
measure P-wave terminal velocity, wherein the P-wave terminal velocity is measured by multiplying the amplitude of the derived V2 in millimeters (mm) and the time duration of the derived V2 negative deflection in milliseconds (ms);
measure Global QRS derivation, wherein measuring the Global QRS derivation comprises looking at the direction and magnitude of the QRS complexes in the derived LI waveform data, LII waveform data, LIII waveform data;
measure a vector of beat and rhythm burdens that includes quantified beat-type and rhythm with ST, PR, QT attributes;
synthesize the measured VAT, the measured P-wave terminal velocity, the measured Global QRS derivation, and the measured vector of beat and rhythm burdens into an Ejection Fraction (EF) biomarker value;
assess the subject's ejection fraction percentage status through comparing the subject's synthesized EF biomarker value with established EF biomarker value norms correlated with specific ejection fraction percentages; and
provide a report to a user of the assessed ejection fraction percentage for the subject.

In some embodiments, the software is configured to implement artificial intelligence (AI) based predictive analysis in assessing the subject's ejection fraction percentage status through comparing the subject's synthesized EF biomarker value with established EF biomarker value norms correlated with specific ejection fraction percentages.

In some embodiments, the ECG has only two leads: LII and LIII, or the ECG has three leads including at least LII and LIII.

In some embodiments, the ECG device is a near-field continuous ECG recording device having three leads plus an on-body ground thereby providing two channels of ECG. In some embodiments, the ECG device is placed onto the mid-center chest region of the subject. In some embodiments, the ECG device includes LII and LIII frontal leads, and one V lead in any off-chest axis position. In some embodiments, ECG device includes LI, LII, and LIII leads in a 12-lead system and one V lead selected from V1, V2, V3, V4, V5, and V6. In some embodiments, the ECG device is configured for placement onto any exterior portion of the subject's body. In some embodiments, the ECG device is configured for placement onto the subject's chest, finger or wrist. In some embodiments, the ECG device is any catheter system which provides at least 1 frontal lead and 1 precordial lead. In some embodiments, the ECG device is any portable, tabletop, bedside telemetry ECG system which provides at least 1 frontal lead and 1 precordial lead. In some embodiments, the ECG device is any episodic or continuous ECG system providing at least 10 seconds of continuous ECG data. In some embodiments, the ECG device is any episodic or continuous ECG system providing at least 10 seconds of continuous ECG in conjunction with algorithm providing rhythm burden. In some embodiments, the ECG device is any episodic or continuous ECG system providing at least 10 seconds of continuous ECG in conjunction with algorithm and Over-Read service providing rhythm burden. In some embodiments, the ECG device is any episodic or continuous ECG system providing at least 10 seconds of continuous ECG in conjunction with Over-Read Service providing rhythm burden.

In some embodiments, the EF is left ventricular ejection fraction (LVEF).

In some embodiments, the subject is a human subject. In some embodiments, the subject is a human subject experiencing or at risk of experiencing a cardiovascular event. In some embodiments, the cardiovascular event is one or more cardiovascular events selected from heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

In some embodiments, the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

In some embodiments, the ECG device is configured to wirelessly transmit via Bluetooth, WI-FI, SD-card, and/or any type or kind of mobile data network.

In some embodiments, the ECG device includes the processor. In some embodiments, the ECG device does not include the processor.

In certain embodiments, the present invention provides a method for characterizing a subject's Ejection Fraction (EF) percentage status, comprising:
providing a system as recited herein;
placing the ECG device onto the subject;
obtaining LII and LIII electrical signal waveform data captured over an extended period of time;
wirelessly transmitting the obtained LII and LIII electrical signal waveform data captured over an extended period of time to the processor via the ECG device;
executing the software to:
   assess the subject's ejection fraction percentage status; and
   provide a report to a user of the assessed ejection fraction percentage for the subject.

In some embodiments, executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value;
assessing the subject's ejection fraction percentage status through comparing the subject's synthesized EF biomarker value with established EF biomarker value norms correlated with specific ejection fraction percentages; and
providing a report to a user of the assessed ejection fraction percentage for the subject.

In some embodiments, a subject's assessed ejection fraction percentage of equal to or greater than 52% for a male and equal to or greater than 54% for a female indicates a normal EF percentage; a subject's assessed ejection fraction percentage of 41% to 51% for a male and 41% to 53% for a female indicates a mildly abnormal EF percentage; a subject's assessed ejection fraction percentage of between 30% to 40% for a male or female indicates a moderately abnormal EF percentage; a subject's assessed ejection fraction percentage of less than 30% for a male or female indicates a severely abnormal EF percentage.

In some embodiments, the subject is a human subject. In some embodiments, the subject is a human subject experiencing or at risk of experiencing a cardiovascular event. In some embodiments, the cardiovascular event is one or more cardiovascular events selected from: heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

In some embodiments, the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 2 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

In certain embodiments, the present invention provides a method for measuring a subject's ECG Ventricular Activation Time (VAT), comprising:
providing a system as recited herein;
placing the ECG device onto the subject;
obtaining LII and LIII electrical signal waveform data captured over an extended period of time;
wirelessly transmitting the obtained LII and LIII electrical signal waveform data captured over an extended period of time to the processor via the ECG device;
executing the software to:
   measure the subject's VAT; and
   provide a report to a user of the measured VAT for the subject.

In some embodiments, executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value.

In some embodiments, the subject is a human subject. In some embodiments, the subject is a human subject experiencing or at risk of experiencing a cardiovascular event. In some embodiments, the cardiovascular event is one or more cardiovascular events selected from: heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

In some embodiments, the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

In certain embodiments, the present invention provides a method for measuring a subject's P-wave Terminal Velocity, comprising:
providing a system as recited herein;
placing the ECG device onto the subject;
obtaining LII and LIII electrical signal waveform data captured over an extended period of time;
wirelessly transmitting the obtained LII and LIII electrical signal waveform data captured over an extended period of time to the processor via the ECG device;
executing the software to:
   measure the subject's P-wave Terminal Velocity; and
   provide a report to a user of the measured P-wave Terminal Velocity for the subject.

In some embodiments, executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value.

In some embodiments, the subject is a human subject. In some embodiments, the subject is a human subject experiencing or at risk of experiencing a cardiovascular event. In some embodiments, the cardiovascular event is one or more cardiovascular events selected from: heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

In some embodiments, the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 2 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

In certain embodiments, the present invention provides a method for treating or preventing reduced ejection fraction (HFrEF) in a subject, comprising:
providing a system recited in claim 1;
assessing the ejection fraction percentage status for the subject as normal, mildly abnormal, moderately abnormal, or severely abnormal;
administering to the subject a therapeutic agent capable of treating or preventing heart failure with reduced ejection fraction if the subject's assessed ejection fraction percentage status is mildly abnormal, moderately abnormal, or severely abnormal.

In some embodiments, a subject's assessed ejection fraction percentage of equal to or greater than 52% for a male and equal to or greater than 54% for a female indicates a normal EF percentage; a subject's assessed ejection fraction percentage of 41% to 51% for a male and 41% to 53% for a female indicates a mildly abnormal EF percentage; a subject's assessed ejection fraction percentage of between 30% to 40% for a male or female indicates a moderately abnormal EF percentage; a subject's assessed ejection fraction percentage of less than 30% for a male or female indicates a severely abnormal EF percentage.

In some embodiments, the therapeutic agent is selected from an ACE inhibitor, an ARB inhibitor, an ARB/neprilysin inhibitor, a beta blocker, an aldosterone antagonist, isosorbide dintrate/hydralazine, a diueretic, and ivabradine.

In some embodiments, the subject is a human subject experiencing or at risk of experiencing heart failure with HFrEF. In some embodiments, the subject is a human subject experiencing or at risk of experiencing a cardiovascular event. In some embodiments, the cardiovascular event is one or more cardiovascular events selected from: heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

In certain embodiments, the present invention provides a method for characterizing a subject's heart failure status, comprising:
providing a system as recited herein;
placing the ECG device onto the subject;
obtaining LII and LIII electrical signal waveform data captured over an extended period of time;
wirelessly transmitting the obtained LII and LIII electrical signal waveform data captured over an extended period of time to the processor via the ECG device;
executing the software to:
   assess the subject's ejection fraction percentage status;
   assess the subject's atrial enlargement status based on the measured P-wave Terminal Velocity; and
   provide a report to a user of the assessed heart failure status for the subject.

In some embodiments, executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value;
assessing the subject's ejection fraction percentage status through comparing the subject's synthesized EF biomarker value with established EF biomarker value norms correlated with specific ejection fraction percentages; and · providing a report to a user of the assessed ejection fraction percentage for the subject.

In some embodiments, the subject is a human subject. In some embodiments, the subject is a human subject experiencing or at risk of experiencing heart failure. In some embodiments, the subject is a human subject experiencing or at risk of experiencing HFrEF or HFpEF.

In some embodiments, the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 2 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

In certain embodiments, the present invention provides a method for estimating an apnea-hypopnea index (AHI) and assessing OSA severity alongside quantification of AFib, Supraventricular, Junctional, Ventricular, Heart Block and Conduction defects with high sensitivity, comprising providing a system recited herein, and implementing the technique recited in Example 4.

### BRIEF DESCRIPTION OF THE DRAWINGS

FIG. 1: (a), a 2-lead wearable ECG is provided representing a miniaturization of Einthoven's Triangle construct in electrocardiograma 2-lead wearable ECG is configured to provide bipolar Lead II and Lead III electrical activity typically obtained using 12-lead ECG systems.

FIG. 2 summarizes the algorithmic workflow and model-chain used in computation and calibration of EF Severity.

FIG. 3 schematically presents the constituents of the EF Biomarker include “Ventricular Activation Time”, “P-Wave Terminal Velocity”, “Global QRS Derivation”, and “Rhythm.”

FIG. 4 shows the diverse heart failure (HF) cohort implemented in Example 1.

FIG. 5A shows a comparison of COR wearable ECG data versus ECHO for various EF severity classes (e.g., each window is a paired point) resulting from implementation of Example 1. FIG. 5B shows 91.4% primary endpoint predictive accuracy for the comparison of COR wearable ECG data versus ECHO for various EF severity classes resulting from implementation of Example 1.

FIG. 6A shows Cohen's Kappa for the overall study resulting from implementation of Example 1. FIG. 6B shows Cohen's Kappa for the “normal” EF severity category resulting from implementation of Example 1. FIG. 6C shows Cohen's Kappa for the “mildly abnormal” EF severity category resulting from implementation of Example 1. FIG. 6D shows Cohen's Kappa for the “moderately abnormal” EF severity category resulting from implementation of Example 1. FIG. 6E shows Cohen's Kappa for the “severely abnormal” EF severity category resulting from implementation of Example 1.

FIG. 7 shows the EF trial sensitivity and specificity results resulting from implementation of Example 1.

FIG. 8 shows an American Society of Echocardiography (ASE) Ejection Fraction severity scale.

FIG. 9 presents a methods schematic implemented in Example 2.

FIG. 10 summarizes the results related to Example 2.

FIG. 11 summarizes Cohen's Kappa for EF, and shows high ECG morphology variability at similar Efs resolved by biomarker.

FIG. 12 shows Leverage COR's broad spectrum rhythm sensitivity and waveform fidelity in LII and all derived leads to detect sub-threshold electrical conduction changes symptomatic of Heart Failure.

FIG. 13 shows results related to Example 3.

FIG. 14 shows results discriminating HFrEF and HFpEF.

FIG. 15 shows additional results related to Example 3.

FIG. 16 summarizes results related to Example 4.

FIG. 17 summarizes results related to Example 4.

FIG. 18 summarizes results related to Example 4.

FIG. 19 summarizes results related to Example 4.

### DETAILED DESCRIPTION

EF measures the heart's ability to pump oxygen-rich blood out to the body (stroke volume) with each beat and is an indicator of heart strength and its muscle health. LVEF is the percentage of oxygen-rich blood pumped out of the heart's left ventricle (LV) to most of the body's organs each time it contracts. LVEF helps determine the severity of dysfunction on the left side of the heart. EF formula equals the amount of blood pumped out of the ventricle with each contraction (stroke volume or SV) divided by the end-diastolic volume (EDV), or the total amount of blood in the ventricle. EF is expressed as a percentage: EF=(SV/EDV)×100. Reduced EF is implicated for diverse myocardial diseases such as ischemia, congenital heart diseases, conduction disorders, infectious diseases, long-term uncontrolled high blood pressure, and granulomatous diseases. The lower the ejection fraction, the weaker the heart's pumping action is, as in the case of people with severe HF.

Heart failure is characterized by dyspnea or exertional limitation due to impairment of ventricular filling or ejection of blood or both. HFrEF occurs when the left ventricular ejection fraction (LVEF) is 40% or less and is accompanied by progressive left ventricular dilatation and adverse cardiac remodeling. As prognosis, therapeutic decisions and therapy effectiveness are often based on LVEF, easy, accurate estimation of instantaneous or spot EF, EF trending, and longitudinal EF excursions has tremendous clinical value. While management of HFrEF has progressed due to drugs, interventional device breakthroughs at a low-cost, low-burden non-invasive at-home measurement are currently unavailable.

Clinical criteria alone are an insufficient basis for the diagnosis of low EF, and the detection of low EF and left ventricular dysfunction cannot rely on clinical signs and symptoms alone, as these may be non-specific and obscured by co-morbidity. Echocardiography (Echo) is the gold standard of cardiac imaging due to its unique ability to non-invasively provide a quantification of cardiac chamber size and function along real-time images of the beating heart (see, Lang R M, et al., J Am Soc Echocardiogr. 2015 January; 28(1):1-39; Beraud, A. Introduction to Transthoracic Echocardiography. Philips Tutorial. http://viewer.zmags.com/publication/9c7aeaf8 #/9c7aeaf8/1). Given its availability and portability advantages, today Echo is preferred for EF spot measurements over other estimators such as semi-invasive cardiac catheterization, and expensive cardiac computed tomography (CT), cardiac MRI and cardiac nuclear stress tests (see, Thomas A Foley, et al., European Cardiology 2012; 8(2): 108-14). However, access to ultrasound echocardiogram in ambulatory and home settings is limited and low-burden EF monitoring would offer tremendous benefit to cardiac patients.

Patch-based continuous, wireless ECG wearables, designed for in-clinic and remote patient monitoring applications are delivering high clinical utility in recording heart activity, detecting arrhythmias and paroxysmal atrial fibrillation (AF, AFib), conduction defects and heart rate variability (HRV) analysis. With a higher diagnostic yield, these reusable, rechargeable, water-proof ECG recording devices are achieving higher compliance rates, patient outcomes and replacing the portable 24-hr Holter monitors for long duration monitoring, adverse cardiac event detection and mobile cardiac telemetry (MCT) applications. Within this device class, FDA-cleared COR ECG XT (K171936) (Cor monitor) is a compact, non-invasive, easy to use device with low-wear burden, high-fidelity ambulatory ECG recording capability. The Cor monitor delivers over 95% analyzable data during wear period for both home and in-clinic use and is capable of detecting up to 31 arrythmias.

Clinical evidence for estimating EF from ECG is based, in part, on the work of Razavipour et al (see, Razavipour F, et al., (2015) J Bioengineer & Biomedical Sci 6:172) where a numerical method for estimation of EF from 12-Lead ECG by calculating the areas and volume under ECG signals is described. A significant correlation (p<0.001) was reported between the values for EF parameters from echocardiography and their numerical results from the areas and volume under the segments of normal ECG signals for 50 subjects by employing trapezoidal, Simpson's, and Boole's rules on three orthogonal planes of 12-lead ECG signal directions and five groups of leads for sagittal, frontal, and transverse planes. In similar vein, Alhamaydeh et al showed poor R wave progression in precordial leads with dominant QS pattern in V3 as a highly predictive feature of reduced LVEF based on a prospective observational cohort study of patients for suspected ACS (see, Alhamaydeh M, et al. J Electrocardiol. 2020 July-August; 61:81-85). O'Neal et al reported several markers detected on the routine 12-lead ECG with high predictive power for future heart failure events (see, O'Neal W T, et al., J Am Heart Assoc. 2017 May 25; 6(6)). They proposed markers of ventricular repolarization and delayed ventricular activation for distinguishing between the future risk of HFrEF and HFpEF. Their findings suggest a role for ECG markers in the personalized risk assessment of heart failure subtypes. Potential for ECG as a noninvasive tool for screening for low EF was strengthened by results from study performed by Chen et al (see, Chen H Y, et al., J Pers Med. 2022 Mar. 13; 12(3):455).

Yao et al identified patients with high likelihood of low EF (see, Yao X, et al., Am Heart J. 2020 January; 219:31-36; Rushlow D R, et al., Mayo Clin Proc. 2022 November; 97(11):2076-2085; Yao X, et al., Nat Med. 2021 May; 27(5):815-819). They employed a deep learning AI algorithm that was prospectively used on routine 12-lead ECG to automatically screen for low ejection fraction based on 12-lead ECG to encourage clinicians to obtain a confirmatory transthoracic echocardiogram (TTE) for previously undiagnosed patients, thereby facilitating early diagnosis and treatment for those at risk of HF. Their primary endpoint was to screen for EF≤50% in adults. Their trial showed that for every 1,000 patients screened, the AI screening yielded five new diagnoses of low ejection fraction over usual care. Building on these results, recent studies have demonstrated an ability to detect LVEF below 40% using single lead ECG recordings to aid in early screening of initial asymptomatic HF with reduced EF (HFrEF). The individual lead was however extracted from a 12-lead ECG to discriminate LVEF above or below the threshold, using echocardiogram as predicate measurement, and achieved sensitivity of 88% and specificity of 74% with Area Under the ROC curve (AUROC) of 0.89 (see, L Guo, et al., European Heart Journal, Volume 42, Issue Supplement_1, October 2021).

In an independent prospective study, Attia et al showed that Electrocardiogram (ECG)-enabled stethoscope (ECG-Scope) acquiring a single-lead ECG during cardiac auscultation may facilitate real-time screening EF≤40% (see, Zachi I Attia, et al., European Heart Journal-Digital Health, Volume 3, Issue 3, September 2022, Pages 373-379). ECG-scope recordings were obtained on a sample size of 100 patients referred for clinically indicated echocardiography, in multiple electrode locations with the patient supine and sitting at the time of the echocardiogram. They trained their AI algorithm for the detection of Left Ventricular Dysfunction (LVSD) using single leads from 12-Lead ECG and validated against ECG-Scope to determine accuracy for low EF detection (≤35%, 40%, or 50%) with respect to body position and lead location. As reported, amongst 100 patients, their best single recording position was V2 with the patient supine [AUC: 0.88 (CI: 0.80-0.97) for EF≤35%, 0.85 (CI: 0.75-0.95) for EF≤40%, and 0.81 (CI: 0.71-0.90) for EF, 50%]. When using an AI model to select the recording automatically, AUC was 0.91 (CI: 0.84-0.97) for EF≤35%, 0.89 (CI: 0.83-0.96).

EF is a volumetric measurement and ECG captures heart's electrical activity. EF is a measure of the percentage of blood leaving the heart each time it contracts, typically measured by volumetric imaging devices such as echocardiography or cardiac MRI, which directly visualize the heart's movement and blood flow. ECG, on the other hand, records the electrical activity of the heart through electrodes placed on the skin surface. The information ECG provides is about the timing and duration of electrical events in the heart, and not directly about the heart's blood flow. The relationship between the electrical activity of the heart and its mechanical function (such as pumping efficiency reflected by EF) is complex and time-varying. Abnormalities in the ECG can suggest potential mechanical dysfunction, but they don't quantitatively measure the volume of blood pumped by the heart. Many heart conditions can non-uniquely alter the ECG signal without necessarily affecting the EF significantly, and vice versa. For instance, a person with a normal EF can still show significant ECG abnormalities due to conditions like electrolyte imbalances, drug effects, or other non-structural cardiac co-morbidities.

Identifying which features of the ECG are most predictive of EF requires deep domain knowledge and sophisticated feature engineering. While deep learning models can automatically learn features from raw data, they still require large amounts of training data and computational resources. The interpretation of ECG signals can be highly variable and depends on subtle features that might not directly correlate with changes in EF. While certain ECG patterns are associated with heart diseases that might reduce EF, such as patterns indicative of previous heart attacks, not all conditions affecting EF produce distinctive ECG changes. ECG morphology used in classical machine learning models and grey or black-box AI models has been shown to fall in the classical trap of locking on fortuitous correlations that do not generalize. ECG signals can be affected by various types of noise and artifacts, including muscle tremors, electrode placement errors, and electrical noise from nearby equipment. These issues can obscure the subtle features that might be indicative of changes in EF.

Developing algorithms or models to predict EF accurately from ECG data requires advanced data analysis techniques, including machine learning and artificial intelligence. These models can be limited by the quality and quantity of the training data, the inherent variability in human physiology, and the complexity of heart diseases to properly annotate. Indeed, there are a significant amount of individual variability in both ECG patterns and EF measurements among people, which can be influenced by factors like age, sex, body size, and underlying health conditions. This heterogeneity and variability renders it challenging to develop a one-size-fits-all approach to predict EF from ECG.

Moreover, ECG signals are susceptible to various types of noise and artifacts, including muscle tremors, interference from other electronic devices, and variations in electrode placement. Small changes or inaccuracies in the ECG data can significantly affect the features extracted for EF prediction, leading to large variations in the predicted EF values. This sensitivity leads to inaccuracies. The relationship between the electrical activity captured by an ECG and the heart's mechanical function (as measured by EF) is complex and not linearly correlated. Small and subtle features in the ECG signal that might be influenced by noise or slight changes could be critical for accurate EF prediction. The complexity and subtlety of this relationship exacerbates the problem's ill-conditioned nature.

The process of feature selection and extraction from ECG signals for predicting EF is crucial. The choice of features and how they are extracted can greatly influence the model's performance. An ill-conditioned problem arises if the model's predictions are highly sensitive to these choices, where minor modifications or the presence of noise in the features can lead to disproportionately large errors in EF prediction. Generally, AI models trained to predict EF from ECG perform satisfactorily on the training data but poorly on unseen data, particularly if the training data do not represent the full range of variability in the population. This lack of generalization can be a symptom of an ill-conditioned problem where the model is overly sensitive to the specific characteristics of the training set. From a computational standpoint, the algorithms used to process ECG data and predict EF must be numerically stable. Ill-conditioned problems are prone to numerical instability, where small computational errors during processing can lead to large discrepancies in the output. Ensuring stability and accuracy in the face of these challenges is a key concern in developing AI models for EF prediction from ECG.

The systems and methods described herein are for characterizing a subject's EF percentage status through use of a wireless and wearable electrocardiogram (ECG) device having LII and LIII leads, and software configured to incorporate data from the ECG device and assess the subject's EF percentage status represent a significant accomplishment over previous techniques and hurdles.

Indeed, experiments described herein demonstrate the feasibility of using only ECG data from a wireless and wearable 2-lead-ECG device having LII and LIII leads to semi-quantitatively categorize EF Severity as per the American Society of Echocardiography (ASE) Scale (EF≥52%; 41-51%; 30-40%; and <30%) over 5-minute time interval and within 15 minutes of starting ECG patch wear (see, Lang R M, et al., J Am Soc Echocardiogr. 2015 January; 28(1):1-39). It was shown that Ejection Fraction and Ejection Fraction severity can be directly and algorithmically estimated from continuous wireless and wearable 2-lead-ECG device having LII and LIII leads waveform analytics and rhythm outputs and is statistically equivalent to EF Severity Category derived from FDA-cleared Echocardiography device output.

As noted, EF is a classification of heart function based on the percentage of blood that is pumped out of the ventricles with each heartbeat. It is an indicator of the heart's mechanical function and efficiency as a pump. Traditionally, EF has been calculated using volumetric measurements obtained through imaging techniques such as echocardiography, cardiac MRI or ventriculography.

Experiments conducted during the course of developing embodiments for the present invention demonstrated 1) direct-from-ECG screening for heart failure with reduced and preserved ejection fraction: 2) a direct-from-ECG Apnea-Hypopnea Index (AHI) for improving AFib and obstructive sleep apnea; and 3) direct-from-ECG Apnea-Hypopnea Index (AHI) for improving AFib and obstructive sleep apnea.

Accordingly, the present invention provides systems and methods for characterizing a subject's ejection fraction (EF) status, heart failure status, and/or sleep apnea status. In particular, described herein are systems and methods for characterizing a subject's EF status, heart failure status, and/or sleep apnea status through use of a wearable electrocardiogram (ECG) device having LII and LIII leads, and software configured to incorporate data from the ECG device and assess the subject's EF status, heart failure status, and/or sleep apnea status.

In the systems and methods described herein, the present invention provides a machine learning enabled model-chain that is configured, upon deployment, to compute EF Severity, a mechanical attribute, from a time-windowed, continuous 2-lead wearable ECG waveform recording (e.g., Peerbridge COR wearable platform) that captures heart's electrical activity in Lead II and Lead III. EF classification accuracy was demonstrated using time windows over 2-minutes, 5-minutes, and 15-minutes of ECG data.

The machine learning enabled model-chain is trained and predictively used on incoming ECG data to algorithmically extract a ECG-based Ejection Fraction Biomarker (“EF Biomarker”) whose numerical constituents have been shown to exhibit both high causality and correlation to heart's mechanical efficacy in the published cardiac literature. The EF Biomarker identifies specific features within the ECG waveform in leads that are physically present in a 2-lead wearable ECG or can be derived (LIII, aVF, aVL, aVR, V2, V3 etc.).

As shown in FIG. 1(a), a 2-lead wearable ECG is provided representing a miniaturization of Einthoven's Triangle construct in electrocardiograma. 1(b)). The 2-lead wearable ECG is configured to provide bipolar Lead II and Lead III electrical activity typically obtained using 12-lead ECG systems.

Einthoven's Triangle is a conceptual geometric model that represents the spatial orientation of the first three leads (Lead I, Lead II, and Lead III) of a 12-lead electrocardiogram (ECG). It allows the heart's electrical activity to be projected onto different axes in the frontal plane. Einthoven Triangle Equations allow computation of LI in COR ECG. Goldberger's Augmented Lead equations allow derivation of aVR, aVL and aVF from the same electrodes as in COR wearable to provide a unipolar view of the heart's electrical activity.

The unique electrode design, geometry, and position on the chest for a 2-lead wearable ECG provides a virtual reference point for approximating precordial (chest) leads V1-V6. Denoted as Wilson's Central Terminal (WCT), this serves as a virtual reference point for the precordial (chest) leads V1-V6 and is derived from the limb leads. A 2-lead wearable ECG enables numerical approximation of precordial lead maxima for each beat detected in Lead II and Lead III. The ability to provide derived frontal and precordial leads, mimicking 12-lead ECG is central to direct-from-ECG determination of EF Severity, as it provides necessary information to compute EF Biomarker constituents which are strongly correlated to volumetric properties of heart's pumping function.

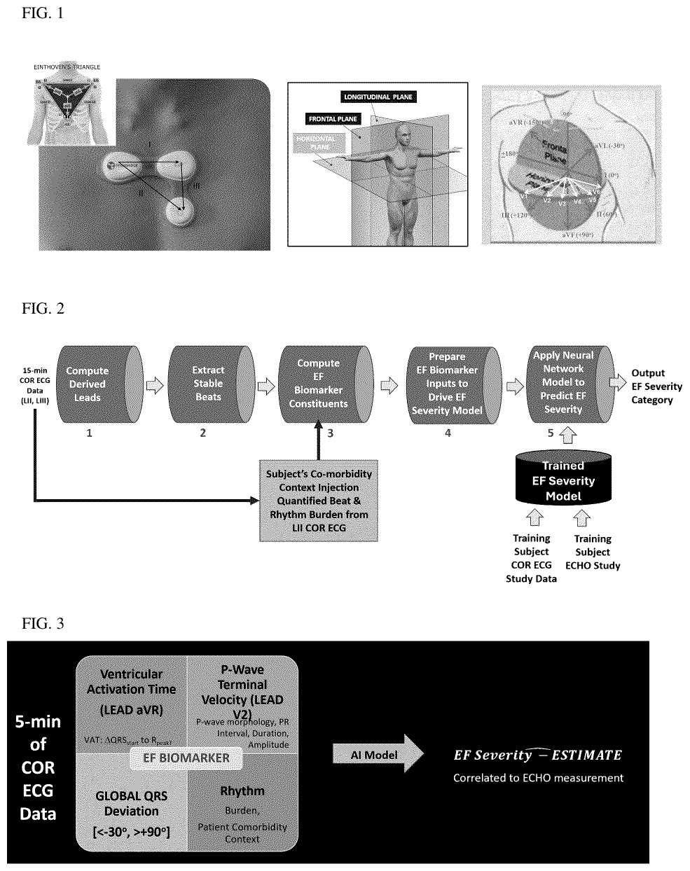

FIG. 2 summarizes the algorithmic workflow and model-chain used in computation and calibration of EF Severity.

Referring to FIG. 2, step 1 pertains to computation of derived leads from COR ECG window raw waveform. This step involves, for example, resampling of 5-minutes of COR ECG LII and LIII waveform data at 200 Hz to 250 Hz, and then using such to derive Lead LI waveform over the window. Derived Lead LI, along with incoming data from LII and LIII is then used to derive the other bipolar frontal leads: aVF, aVR and aVL over the same time window. Once the frontal leads are derived-a spatial vector cardiography transform is used to estimate Wilson Central Terminal Reference potential that is further used to approximate the beat-by-beat maxima for pre-cordial leads V1 thru V6.

Referring to FIG. 2, step 2 pertains to extraction of stable beats in ambulatory COR ECG. Time synchronized LII and LIII windowed data, and corresponding records of all derived leads are filtered to remove motion and other artifacts. Spectral entropy for each beat in the 5-minute window is calculated to remove all beats above pre-set threshold model. Consequently, any derived beat associated with LII or LIII beat with spectral entropy outside the threshold is also filtered out. PQRST scaffolding (PR, QT, ST interval) etc. are computed for the surviving stable beats. Windowed HRV is computed to ensure no artifacts. If artifacts are observed, spectral entropy threshold is reduced by 10% to remove additional beats. The process is repeated until only stable beats remain in the input set.

Referring to FIG. 2, step 3 pertains to computation of constituents of the EF Biomarker using all leads (LII, LIII and all derived Leads) (see, FIG. 3). As shown in FIG. 3, the constituents of the EF Biomarker include “Ventricular Activation Time”, “P-Wave Terminal Velocity”, “Global QRS Derivation”, and “Rhythm.”

Referring to FIG. 3, Ventricular Activation Time (VAT) for derived lead a VR, refers to the time interval required for the electrical activation to spread through the ventricles. Specifically, in lead aVR, VAT is measured from the onset of the QRS complex to the peak of the R wave. This measurement reflects the conduction time through the ventricles, which can be indicative of ventricular hypertrophy or conduction abnormalities that can lead to changes in EF severity.

Referring to FIG. 3, P-wave terminal velocity in lead V2 analytic is focused on the terminal portion of the P-wave, which reflects atrial depolarization, particularly of the left atrium. In lead V2, which is one of the precordial (chest) leads positioned in the fourth intercostal space to the left of the sternum, the P-wave can exhibit a biphasic pattern with an initial positive component followed by a negative component. The negative component represents the depolarization of the left atrium that travels away from lead V2's viewpoint. The “terminal velocity” of the P-wave in this context typically refers to the depth and duration of the terminal (negative) portion of the P-wave in lead V2. This is quantitatively assessed by:
Depth: The amplitude of the negative deflection in millimeters (mm); and
Duration: The time duration of the negative deflection in milliseconds (ms)
P-wave Terminal Force=Depth (mm)*Duration (ms).
   Abnormalities in the P-wave terminal force, such as an increased depth or prolonged duration in lead V2, has been shown to be indicative of left atrial enlargement or increased left atrial pressure and seen in patients with EF abnormalities.

Referring to FIG. 3, Global QRS deviation in an ECG, often referred to as “axis deviation,” refers to the overall direction of the heart's electrical activity during ventricular depolarization, as represented by the QRS complex. “Global” implies that this reflects the sum total of all electrical forces in the heart during ventricular depolarization, rather than the activity in a single part of the heart. In the context of leads I, II, and III (the limb leads), the QRS axis is particularly informative as these leads provide views of the heart's electrical activity in the frontal plane:
Lead I records electrical activity between the right arm and left arm electrodes.
Lead II records activity between the right arm and left leg electrodes.
Lead III records activity between the left arm and left leg electrodes.
   The global QRS axis is typically determined by looking at the direction and magnitude of the QRS complexes in these leads.

Referring to FIG. 3, vector of beat and rhythm burdens that includes quantified beat-type and rhythm is a vector includes ST, PR, QT attributes. P-Wave Terminal Velocity is computed using a trained neural network whose parameters include P-wave amplitudes for derived leads V2, V3, V5 and V6.

Referring again to FIG. 2, the computed constituents of the EF Biomarker (e.g., “Ventricular Activation Time”, “P-Wave Terminal Velocity”, “Global QRS Derivation”, and “Rhythm”), are synthesized for preparing a normalized input (EF Biomarker vector) that is used to drive a EF Model trained offline using paired 5-min ECG windowed samples and ECHO categories for the same subject. Paired samples used in building the neural network classifier model are from subjects in a resting sitting position where the COR ECG window is within an hour of getting ECHO study completed or where the subject wore COR ECG and had an ECHO study done using FDA cleared Ultrasound device. EF % values obtained in ECHO study using Simpson's Biplane method are converted to EF levels on the ASE scale and used in training. Training is done to construct a mapping between EF Biomarker inputs and EF Category Outputs.

EF Biomarker input vector for all constituents except Beat & Rhythm burden are synthesized at the beat level. All attributes of the EF Biomarker vectors for all surviving beats in a 5-minute window are then averaged to form a single input vector for the entire window.

EF Biomarker for the entire 5-minute window is then used as input to a trained neural network EF classification model to output a single EF value for the subject for that window.

In certain aspects, a system for characterizing a subject's EF percentage status is provided, comprising an electrocardiogram (ECG) device and a processor. In some aspects, the EF is left ventricular ejection fraction (LVEF). In some aspects, the subject is a mammal. In some aspects, the subject is a human subject. In some aspects, the subject is a human subject having or at risk of developing a cardiovascular condition (e.g., heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.).

Such systems are not limited to a particular type of ECG device.

In some aspects, the ECG device is 1) wearable or portable, 2) has LII and LIII leads, 3) is configured upon placement onto a subject to capture LII and LIII electrical signal waveform data from the subject via the LII and LIII leads over an extended period of time, and 4) is configured to wirelessly transmit LII and LIII electrical signal waveform data from the subject captured over an extended period of time to a processor.

In some aspects, the ECG device has only two leads: LII and LIII, or has three leads including at least LII and LIII. In some aspects, the ECG device is a near-field continuous ECG recording device having three leads plus an on-body ground thereby providing two channels of ECG. In some aspects, the ECG device is placed onto the mid-center chest region of the subject. In some aspects, the ECG device includes LII and LIII frontal leads, and one V lead in any off-chest axis position. In some aspects, the ECG device includes LI, LII, and LIII leads in a 12-lead system and one V lead selected from V1, V2, V3, V4, V5, and V6. In some aspects, the ECG device is configured for placement onto any exterior portion of the subject's body. In some aspects, the ECG device is configured for placement onto the subject's chest, finger or wrist. In some aspects, the ECG device is any catheter system which provides at least 1 frontal lead and 1 precordial lead. In some aspects, the ECG device is any portable, tabletop, bedside telemetry ECG system which provides at least 1 frontal lead and 1 precordial lead. In some aspects, the ECG device is any episodic or continuous ECG system providing at least 10 seconds of continuous ECG data. In some aspects, the ECG device is any episodic or continuous ECG system providing at least 10 seconds of continuous ECG in conjunction with algorithm providing rhythm burden. In some aspects, the ECG device is any episodic or continuous ECG system providing at least 10 seconds of continuous ECG in conjunction with algorithm and Over-Read service providing rhythm burden. In some aspects, the ECG device is any episodic or continuous ECG system providing at least 10 seconds of continuous ECG in conjunction with Over-Read Service providing rhythm burden.

In some aspects, the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 2 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

In some aspects, the ECG device is configured to wirelessly transmit via Bluetooth, WI-FI, SD-card, and/or any type or kind of mobile data network.

In some aspects, the ECG device is described in US20190313968A1, WO2019073288, KR102309022B1, U.S. Pat. Nos. 5,191,891A, 6,871,089B2, US20120191147A1, KR101649445B1, US20150297433A1A, KR20130137327A, DE4217388A1, FR3041876A1, US20110034784A1, https://ieeexplore.ieee.org/abstract/document/4535543, https://ieeexplore.ieee.org/document/6122530, https://ieeexplore.ieee.org/document/1621147, https://ieeexplore.ieee.org/abstract/document/4201276, and/or http://citeseerx.ist.psu.edu/viewdoc/download?doi=10.1.1.639.9471rep=repltype=pdf.

In some aspects, the ECG device is described in US20230240614A1, WO2018017508A1, U.S. Pat. No. 9,681,825B2, and/or U.S. Pat. No. 9,675,264B2.

Such systems are not limited to a particular type of processor.

In some aspects, the processor comprises software that, when executed, causes the processor to manually or automatically receive LII and LIII electrical signal waveform data captured over an extended period of time from a subject wirelessly transmitted from a ECG device; and utilize the received LII and LIII electrical signal waveform data captured over an extended period of time to 1) assess the subject's ejection fraction percentage status; and 2) provide a report to a user of the assessed ejection fraction percentage for the subject.

In some aspects, the software, when executed, is configured to accomplish one or more of the following:
resample the LII and LIII electrical signal waveform data captured over an extended period of time to 250 Hz;
derive LI waveform with resampled LI and LII waveform data;
utilize the derived LI waveform and resampled LII and LIII waveform data to derive the following other bipolar frontal leads: aVF, aVR and aVL;
estimate a Wilson Central Terminal Reference potential with a spatial vector cardiography transform;
approximate a beat-by-beat maxima for pre-cordial leads V1 thru V6 with the spatial vector cardiography transform;
filter the derived LI waveform data, LII waveform data, LIII waveform data, aVF data, aVR data, aVL data, Wilson Central Terminal Reference potential, and V1 thru V6 data to remove motion (and other) artifacts;
calculate a spectral entropy for each heartbeat over the time period and remove all heartbeats above a pre-set threshold model;
compute PR, QT, ST interval for remaining stable heart-beats;
compute windowed HRV;
measure ventricular activation time (VAT) for derived lead aVR, wherein the VAT is measured from the onset of the QRS complex to the peak of the R wave;
measure P-wave terminal velocity, wherein the P-wave terminal velocity is measured by multiplying the amplitude of the derived V2 in millimeters (mm) and the time duration of the derived V2 negative deflection in milliseconds (ms);
measure Global QRS derivation, wherein measuring the Global QRS derivation comprises looking at the direction and magnitude of the QRS complexes in the derived LI waveform data, LII waveform data, LIII waveform data;
measure a vector of beat and rhythm burdens that includes quantified beat-type and rhythm with ST, PR, QT attributes;
synthesize the measured VAT, the measured P-wave terminal velocity, the measured Global QRS derivation, and the measured vector of beat and rhythm burdens into an Ejection Fraction (EF) biomarker value;
assess the subject's ejection fraction percentage status through comparing the subject's synthesized EF biomarker value with established EF biomarker value norms correlated with specific ejection fraction percentages; and
provide a report to a user of the assessed ejection fraction percentage for the subject.

In some aspects, the software is configured to implement artificial intelligence (AI) based predictive analysis in assessing the subject's ejection fraction percentage status through comparing the subject's synthesized EF biomarker value with established EF biomarker value norms correlated with specific ejection fraction percentages.

In some aspects, the processor is further configured to display a provided report. In some aspects, the ECG device includes the processor. In some aspects, the ECG device does not include the processor.

In certain aspects, methods for characterizing a subject's Ejection Fraction (EF) percentage status are provided, wherein the method comprises providing a system for characterizing a subject's EF percentage status as described herein, and 1) placing the ECG device onto the subject; 2) obtaining LII and LIII electrical signal waveform data captured over an extended period of time; 3) wirelessly transmitting the obtained LII and LIII electrical signal waveform data captured over an extended period of time to the processor via the ECG device; and 4) executing the software to assess the subject's ejection fraction percentage status, and provide a report to a user of the assessed ejection fraction percentage for the subject.

In some aspects, executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value;
assessing the subject's ejection fraction percentage status through comparing the subject's synthesized EF biomarker value with established EF biomarker value norms correlated with specific ejection fraction percentages; and
providing a report to a user of the assessed ejection fraction percentage for the subject.

In some aspects, a subject's assessed ejection fraction percentage of equal to or greater than 52% for a male and equal to or greater than 54% for a female indicates a normal EF percentage. In some aspects, a subject's assessed ejection fraction percentage of 41% to 51% for a male and 41% to 53% for a female indicates a mildly abnormal EF percentage. In some aspects, a subject's assessed ejection fraction percentage of between 30% to 40% for a male or female indicates a moderately abnormal EF percentage. In some aspects, a subject's assessed ejection fraction percentage of less than 30% for a male or female indicates a severely abnormal EF percentage. In some aspects, a subject's assessed ejection fraction percentage is categorized per the American Society of Echocardiography (ASE) Scale (EF≥52%; 41-51%; 30-40%; and <30%) over 5-minute time interval and within 15 minutes of starting ECG patch wear (see, Lang R M, et al., J Am Soc Echocardiogr. 2015 January; 28(1):1-39).

In certain aspects, methods for measuring a subject's ECG Ventricular Activation Time (VAT) are provided, wherein the method comprises 1) providing a system as described herein, 2) placing the ECG device onto the subject; 3) obtaining LII and LIII electrical signal waveform data captured over an extended period of time; 4) wirelessly transmitting the obtained LII and LIII electrical signal waveform data captured over an extended period of time to the processor via the ECG device; and 6) executing the software to measure the subject's VAT, and provide a report to a user of the measured VAT for the subject.

In some aspects, executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value;
assessing the subject's ejection fraction percentage status through comparing the subject's synthesized EF biomarker value with established EF biomarker value norms correlated with specific ejection fraction percentages; and
providing a report to a user of the assessed ejection fraction percentage for the subject.

In certain aspects, methods for measuring a subject's P-wave Terminal Velocity 1) providing a system as described herein, 2) placing the ECG device onto the subject; 3) obtaining LII and LIII electrical signal waveform data captured over an extended period of time; 4) wirelessly transmitting the obtained LII and LIII electrical signal waveform data captured over an extended period of time to the processor via the ECG device; and 6) executing the software to measure the subject's P-wave Terminal Velocity, and provide a report to a user of the measured P-wave Terminal Velocity for the subject.

In some aspects, executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value;
assessing the subject's ejection fraction percentage status through comparing the subject's synthesized EF biomarker value with established EF biomarker value norms correlated with specific ejection fraction percentages; and
providing a report to a user of the assessed ejection fraction percentage for the subject.

The systems and methods described herein (e.g., for characterizing a subject's EF percentage status through use of a ECG device having LII and LIII leads, and software configured to incorporate data from the ECG device and assess the subject's EF percentage status) are designed to measure EF and EF Severity on-demand anywhere that a subject (e.g., a human patient) may be, whether in the clinic, hospital, or at home. Evaluations can take a low as minutes, do not require specialized personnel or additional equipment, and are highly cost effective.

The systems and methods described herein (e.g., for characterizing a subject's EF percentage status through use of a ECG device having LII and LIII leads, and software configured to incorporate data from the ECG device and assess the subject's EF percentage status) have the potential to revolutionize EF percentage assessment. Measurements can be made in as little as 2 to 15 minutes that can be applied in the clinic, emergency department, or patient's home without the need for specialized technicians. This would allow point of care screening at scale.

In certain embodiments, the present invention provides a method for characterizing a subject's heart failure status, comprising:
providing a system as recited herein;
placing the ECG device onto the subject;
obtaining LII and LIII electrical signal waveform data captured over an extended period of time;
wirelessly transmitting the obtained LII and LIII electrical signal waveform data captured over an extended period of time to the processor via the ECG device; and
executing the software to:
   assess the subject's ejection fraction percentage status;
   assess the subject's atrial enlargement status based on the measured P-wave Terminal Velocity; and
   provide a report to a user of the assessed heart failure status for the subject.

In some embodiments, executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value;
assessing the subject's ejection fraction percentage status through comparing the subject's synthesized EF biomarker value with established EF biomarker value norms correlated with specific ejection fraction percentages; and
providing a report to a user of the assessed ejection fraction percentage for the subject.

In some embodiments, the subject is a human subject. In some embodiments, the subject is a human subject experiencing or at risk of experiencing heart failure. In some embodiments, the subject is a human subject experiencing or at risk of experiencing HFrEF or HFpEF.

In some embodiments, the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 2 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

In certain embodiments, the present invention provides a method for estimating an apnea-hypopnea index (AHI) and assessing OSA severity alongside quantification of AFib, Supraventricular, Junctional, Ventricular, Heart Block and Conduction defects with high sensitivity with the system recited in claim 1 and the steps recited in Example 4.

In some aspects, the processor is any type or kind of computing device (e.g., laptop, desktops, workstation, personal digital assistant, server, blade server, mainframe, and other appropriate computers). In some aspects, the processor is a part of or functions within a computing device (e.g., laptop, desktops, workstation, personal digital assistant, server, blade server, mainframe, and other appropriate computers). In some aspects, the computing devices is intended to represent various forms of mobile devices, such as personal digital assistants, cellular telephones, smartphones, and other similar computing devices. In some aspects, the computing device can include Universal Serial Bus (USB) flash drives. The USB flash drives may store operating systems and other applications. The USB flash drives can include input/output components, such as a wireless transmitter or USB connector that may be inserted into a USB port of another computing device.

In some aspects, the memory stores information within the computing device and/or processor. In one implementation, the memory is a volatile memory unit or units. In another implementation, the memory is a non-volatile memory unit or units. The memory may also be another form of computer-readable medium, such as a magnetic or optical disk.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

### EXAMPLES

The following examples are intended only to illustrate methods and embodiments in accordance with the invention, and as such should not be construed as imposing limitations upon the claims.

### Example 1

This example provides results related to a Direct-From-ECG EF Severity study.

An EF feasibility study was completed with a cohort of 36 subjects. Prediction of EF severity using COR ECG-only was statistically non-inferior to ECHO outputs on American Society of Echocardiography (ASE) severity scale (e.g., using only a 5-minute of ECG data window) (e.g., within 5-minutes of ECG wear). EF severity was predicted at a 91.4% accuracy with AUC of 0.94 (65 paired test points, with 267 5-minute windows). ECG windows were randomly drawn from 36 patients with two ECHO visits. Visit one was ECHO+60 minutes of COR ECG data where ECG study started within 30 minutes of getting ECHO EF measurement. Visit 2 was ECHO taken with COR ECG device on study participant's chest and recording data. Paired point 60 minutes before ECCHO timestamp on study participant's ECHO report or within 60 minutes following ECHO timestamp. T-60 minutes<T: ECHO Time Stamp on study participant ECHO Report<T+60 minutes.

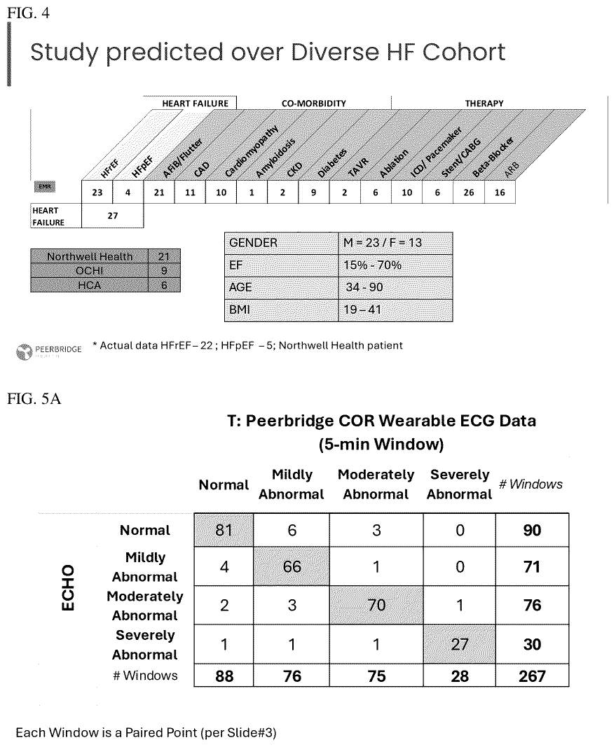

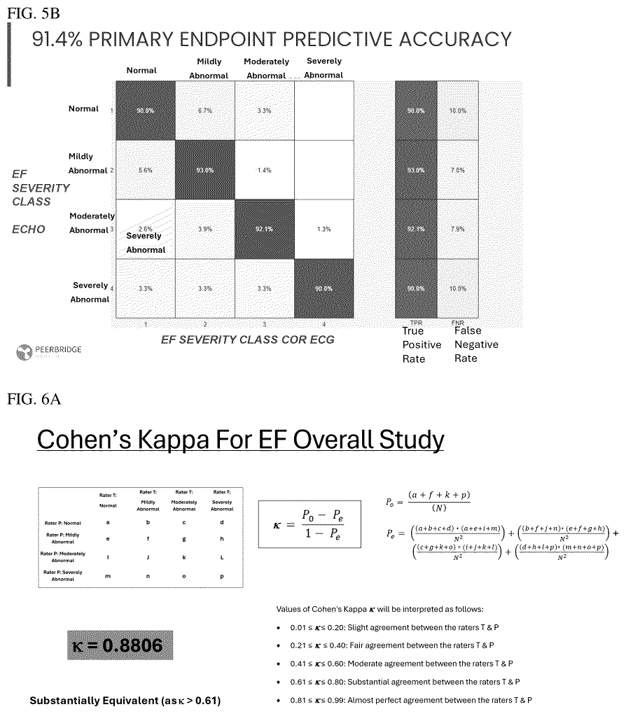

FIG. 4 shows the diverse heart failure (HF) cohort implemented in Example 1.

FIG. 5A shows a comparison of COR wearable ECG data versus ECHO for various EF severity classes (e.g., each window is a paired point) resulting from implementation of Example 1. FIG. 5B shows 91.4% primary endpoint predictive accuracy for the comparison of COR wearable ECG data versus ECHO for various EF severity classes resulting from implementation of Example 1.

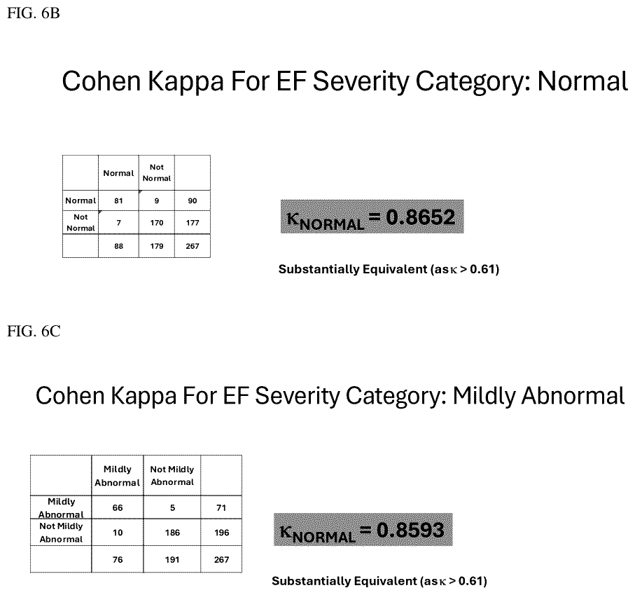

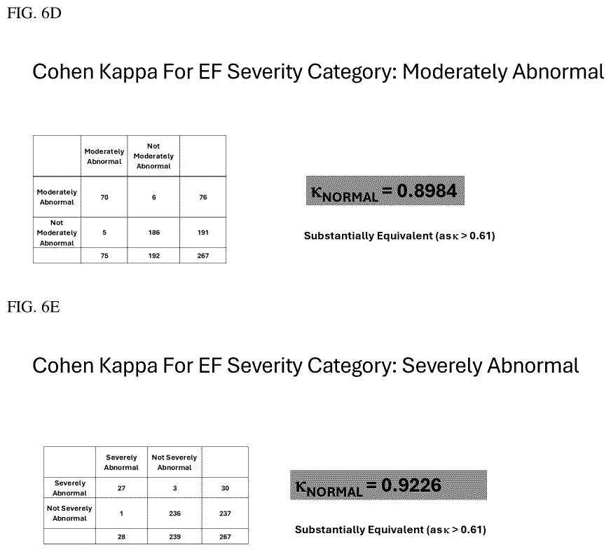

FIG. 6A shows Cohen's Kappa for the overall study resulting from implementation of Example 1. FIG. 6B shows Cohen's Kappa for the “normal” EF severity category resulting from implementation of Example 1. FIG. 6C shows Cohen's Kappa for the “mildly abnormal” EF severity category resulting from implementation of Example 1. FIG. 6D shows Cohen's Kappa for the “moderately abnormal” EF severity category resulting from implementation of Example 1. FIG. 6E shows Cohen's Kappa for the “severely abnormal” EF severity category resulting from implementation of Example 1.

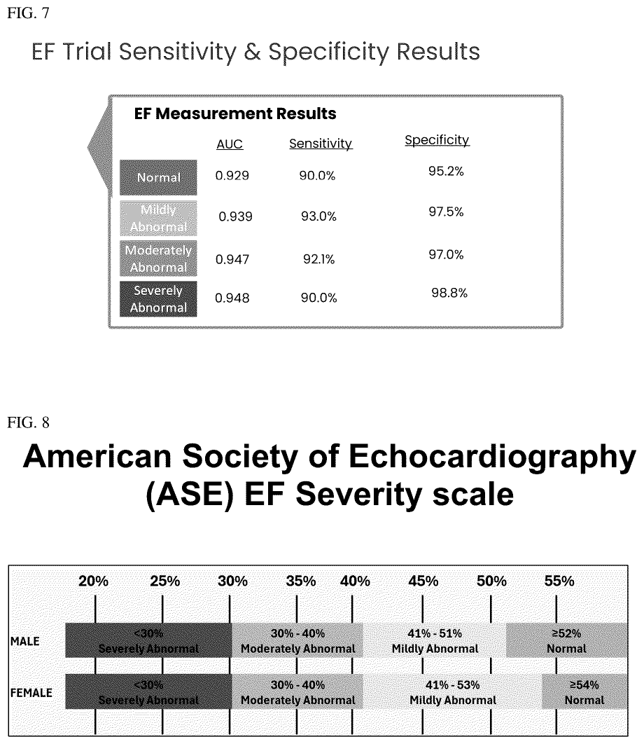

FIG. 7 shows the EF trial sensitivity and specificity results resulting from implementation of Example 1.

### Example 2

This example demonstrates direct-from-ECG ejection fraction severity for managing heart failure in patients with multiple co-morbidities.

Currently, 12-lead ECG and wearables only allow heart failure (HF) screening of subjects above vs. below a threshold (e.g., LVEF<40%). Larger datasets or algorithmic variants have not yielded higher EF fidelity due to distortion of ECG morphology by comorbidities (direct-from-ECG EF determination is an ill-posed and ill-conditioned inverse problem).

Experiments were conducted that resulted in the development of an alternate algorithm for ECG wearables to detect LV dysfunction and provide EF stratification in alignment with the American Society of Echocardiography (ASE) Ejection Fraction severity scale (see, FIG. 8).

Indeed, such experiments were conducted to validate a novel AI-enabled ECG-derived biomarker for predicting EF severity in a prospective, multicenter trial against ECHO reference and demonstrate predictive power with 3-5 orders of magnitude lesser training data compared to deep learning.

An objective of these experiments was to demonstrate EF severity agreement. For example, an objective was to validate ambulatory ECG against ECHO in a multi-center clinical trial (ASE Scale) (e.g., enabled by Peerbridge COR 3-lead ECG patch based on near-field Einthoven's Triangle). An objective was to establish a new, physiologically-based, demographic-independent ECG biomarker for EF prediction.

An objective of these experiments was to develop an on-demand EF severity assessment with low burden (e.g., quickly assess EF severity with 5 to 15 minutes of ECG data) (e.g., applicable in all settings: clinic, hospital, home).

An object of these experiments was to enhance clinical utility of ECG patches (e.g., use ambulatory continuous ECG wearable to detect longitudinal changes in cardiac function through volumetric analytics).

39 subjects with NYHA Class II/III HF or breathlessness/palpitations received were monitored for 4-7 days with an FDA-cleared COR™ chest patch that provided 3-lead continuous ECG. COR, a miniaturization of Einthoven triangle topology, using only LII & LIII provides all 6 frontal leads. A unique precordial trajectory approximation was used to estimate V1-V6 peak amplitudes over a series of 5-minute windows. Beat-to-beat variability, ambulatory artifacts were filtered out, and features of interest, such as QRS duration and ventricular activation time, and known cardiac comorbidities, were provided to a calibrated AI-model to predict EF severity over each time window. Patients also received 2 echocardiograms (ECHO) over the study (one with device and one without). ECG-derived EF severity was then compared to ECHO-derived EF severity (the gold standard) to assess model's predictive power.

The developed model yielded 91.4% accuracy with an area under the curve of 0.94 in predicting EF severity using only 5 minutes of ECG data on 36 in-protocol participants that included 23 with HFrEF and 4 with HFpEF. 50% of paired points were used for training and 50% for prediction. For each ASE severity category (LVEF≥52%, 41-52%, 30-40%, <30%), sensitivity was ≥90% (90%-93%) and specificity was ≥93% (93%-95%). Participant EF ranged from 15% to 70%, age from 34-90, and BMI from 19-41. Cohort comorbidities included AFib, CAD, Cardiomyopathy, CKD, and Diabetes; with therapies ranging from TAVR, ICD, ablation, and CABG/stent.

Such experiments demonstrate that accurate direct-from-ECG EF severity determination can expand access to HF diagnosis at low cost and enable early detection and intervention for a broad growing population at risk for LV dysfunction.

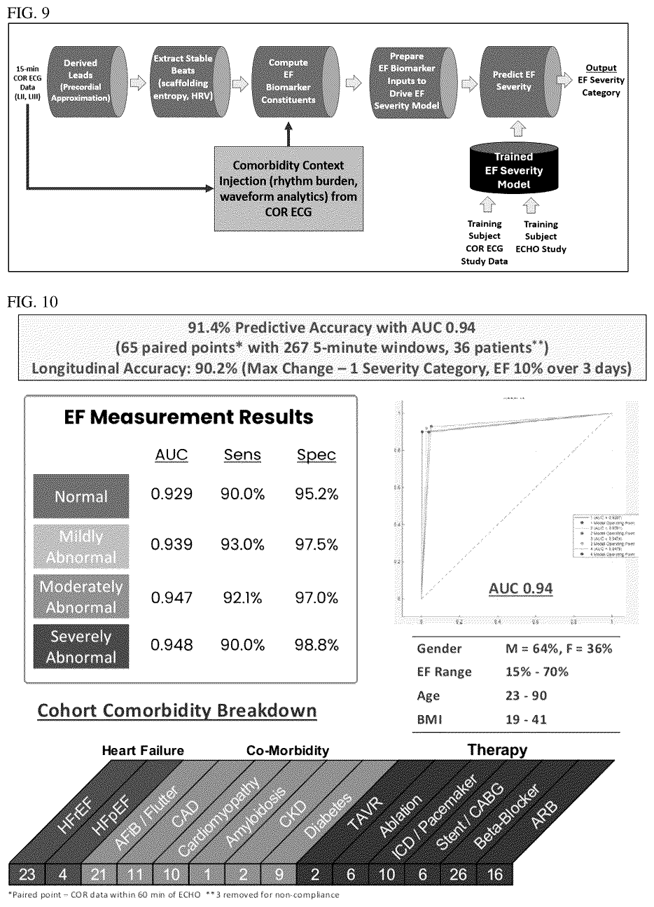

FIG. 9 presents a methods schematic implemented in Example 2.

FIG. 10 summarizes the results related to Example 2.

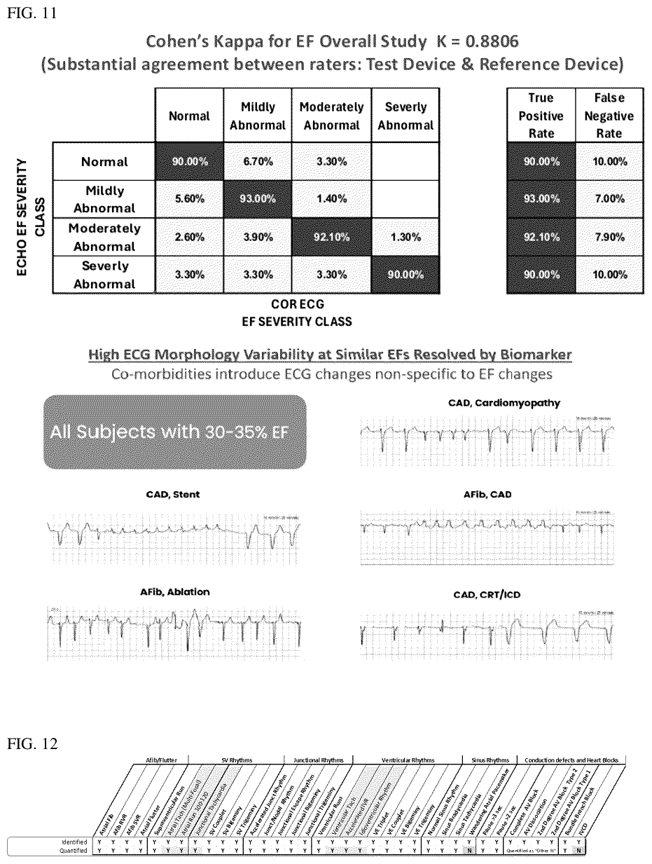

FIG. 11 summarizes Cohen's Kappa for EF, and shows high ECG morphology variability at similar Efs resolved by biomarker.

The following conclusions resulted from Example 2:
COR® ECG enables on-demand EF severity prediction, matching the accuracy of ECHO across all ASE severity categories predictive accuracy of 91.4% using just 5 minutes of ECG data;
100× less training data required compared to typical deep learning models;
Demographic-agnostic with novel ECG-derived patient context injection;
Enables early detection of LV dysfunction and low EF in all care settings in patients undergoing ECG Holter/XT/MCT for other indications;
Broadly applicable-supports instantaneous, continuous, and longitudinal EF severity excursion assessment, facilitating the design and monitoring of cardiac treatments and rehabilitation programs
Starting 800 subject, prospective multicenter trial for EF Severity software as a medical device (SAMD) label.

### Example 3

This example demonstrates direct-from-ECG screening for heart failure with reduced and preserved ejection fraction.

Early heart failure (HF) diagnosis would likely improve clinical outcomes, however over 60% of patients are first diagnosed with HF in the emergency department. These patients typically present with severe HF symptoms arising from the misinterpretation of earlier signs, lack of prior medical engagement and limited access to care.

ECG-based HF screening has been limited to determination of whether the left ventricular ejection fraction (LVEF) is greater than or less than a threshold value such as 40%. False positives and false negatives remain problematic due to confounding influence of co-morbidities on LVEF, signal quality, and high HF-symptom bias in AI models.

Building on the body of work on subtle p-wave morphology changes in HF, experiments were conducted that resulted in a robust classifier for HF that does not require symptom inputs.

Indeed, experiments were conducted to validate a novel direct-from-ECG marker for HF screening in a prospective, multicenter trial evaluated against EHR-derived HF diagnosis, and the ability to differentiate HF with preserved ejection fraction (HFrEF) from HF with reduced ejection fraction (HFpEF) was assessed.

Objectives for these experiments included:
Demonstrate direct-from-ECG HF Screening
   Validate Peerbridge COR® ECG HF-detection against EHR-tagged subject's HF diagnosis in a multi-center prospective, clinical trial
   Establish a new, symptom-independent ECG bio-marker for HF detection
On-Demand, multiplexed HF screening and EF Severity with low burden
   Quickly assess HF risk with 5 minutes of continuous ECG data
   Applicable in all settings: clinic, hospital, or home
Ability to discriminate HFrEF with HFpEF using COR® ECG.

A 34-subject feasibility trial was conducted that included 23 patients with HFrEF, 4 with HFpEF, and 7 without electronic health record (EHR) symptoms of HF. Subjects were monitored using COR™, an ambulatory ECG patch based on Einthoven's triangle orientation. Starting with only Lead II and Lead III amplitudes, 6 frontal leads and 4 precordial leads were derived using a spatial vector cardiography model unique to COR. P-wave properties from derived leads were fed to a neural network model chain trained to differentiate patients with HF from no-HF and HFrEF from HFpEF. QRS duration and ventricular activation time were used to predict numerical EF % for use in making HF calls. Patients also received two echocardiograms that were used to calibrate the numerical EF % prediction AI model. FIG. 12 shows Leverage COR's broad spectrum rhythm sensitivity and waveform fidelity in LII and all derived leads to detect sub-threshold electrical conduction changes symptomatic of Heart Failure.

Eighty percent of paired points were used for training and 20% for prediction. Predicted diagnosis of HF was scored against subject's HF clinical status as stated in the EHR. HF calls could be made within 10-min of COR wear and using only 5 min of ECG data. COR HF marker demonstrated a 95.7% predictive accuracy, 4.3% false discovery rate, with an AUC of 0.97 in classifying subjects as HF vs. no HF. HFrEF was successfully differentiated from HFpEF in 26/27 subjects.

The low burden, ECG-derived HF biomarker demonstrates promise for broad screening of individuals with both reduced and preserved LVEF. COR AECG's p-wave fidelity, longitudinal stability and Einthoven triangle enabled derived leads are key to its predictive power.

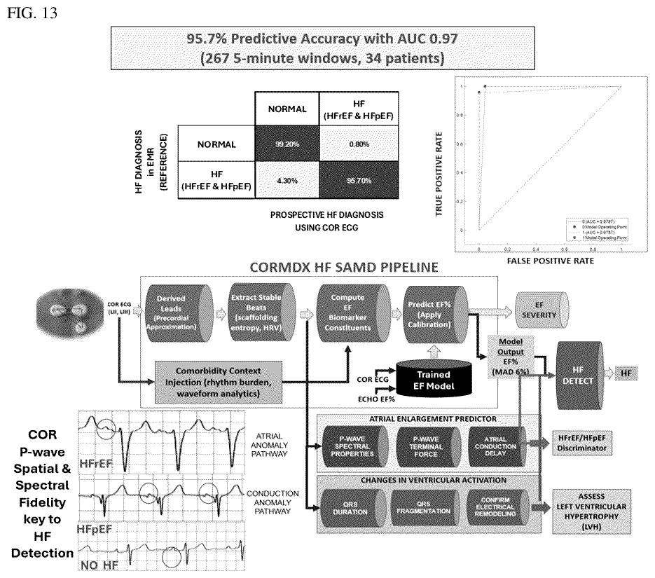

FIG. 13 shows results related to Example 3.

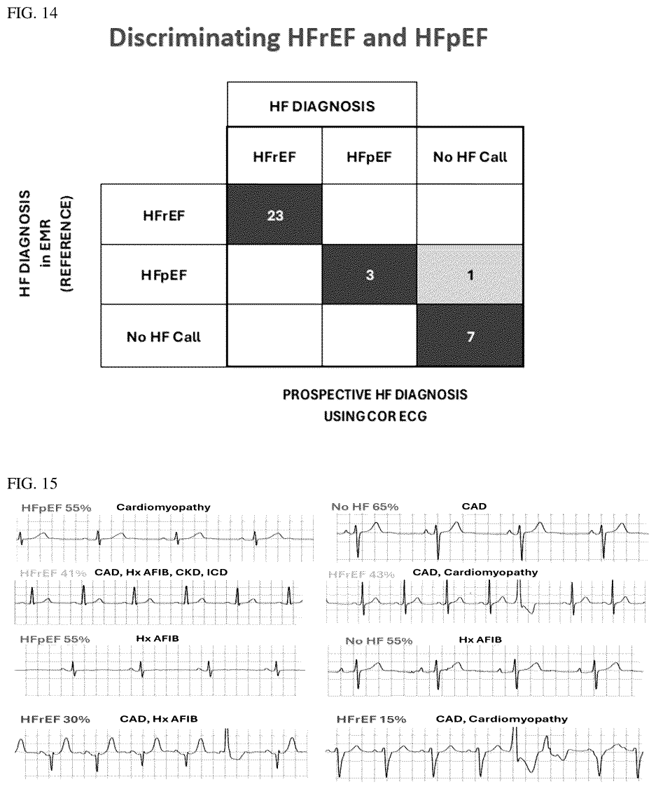

FIG. 14 shows results discriminating HFrEF and HFpEF.

FIG. 15 shows additional results related to Example 3.

The following conclusions resulted from Example 3:
COR® ECG demonstrates on-demand HF detection with 95.7% accuracy in both symptomatic and asymptomatic patients using just 5 minutes of data, as shown in a feasibility trial.
A novel biomarker, grounded in well-established HF causality pathways, functions independently of symptom bias, improving on traditional methods.
HF biomarker effectively screens for HFpEF and differentiates from HFrEF, enhancing the capabilities of existing ECG models.
Early HF screening using ECG offers significant benefits due to its affordability, ease of access, convenience, and potential to lower mortality and morbidity in resource-limited settings.
Integrating multiplexed assessments of atrial function and ventricular degradation with HF detection broadens the biomarker's clinical applicability.

### Example 4

This example demonstrates a direct-from-ECG Apnea-Hypopnea Index (AHI) for improving AFib and obstructive sleep apnea.

Nearly 50% of patients with AFib have comorbid obstructive sleep apnea (OSA), and 75% of OSA patients report some form of arrhythmia. This strong coupling is attributed to pronounced effects of severe OSA on the heart, including greater fluctuations in intrathoracic pressure and cardiac afterload, oxidative stress and remodeling from intermittent hypoxia; and altered autonomic regulation due to increased sympathetic nervous system activity. With 80% OSA cases undiagnosed, untreated OSA increases the likelihood of poor outcomes for AFib patients, including failed treatment, heart failure, stroke, and sudden cardiac death. OSA severity is diagnosed based on AHI: accepted as a compliance, efficacy, and effectiveness metric for CPAP and other OSA therapies. Ambulatory ECG wearables offer an attractive alternate Home Sleep Test (HST) platform for AHI due to their low burden, low cost, multi-night, simultaneous cardiac and respiratory assessment

However, direct-from-ECG AHI estimation is very challenging due to difficulty in distinguishing shallow breathing changes (hypopneas) from subtle physiological variations and individual differences in ECG-derived respiration (EDR) waveforms, compounded by signal noise, artifacts, complex heart-respiratory interactions, algorithmic limitations, and the impact of comorbid.

Objectives for these experiments included:
Demonstrate AHI Agreement with WatchPAT® HST for Sleep Apnea
   Prospective trial with one sleep night
   Diverse cohort with cardio-pulmonary and other co-morbidities
Validate:
   EDR-derived marker for hypopnea detection, adaptive to individual's breathing depth: emulating function that has typically required respiratory inductive plethysmography (RIP), optical, strain, acoustic, or vibration sensors in HST
   Respiratory Effort-Related Arousals (RERAs) in COR LII and LIII ECG following apnea events to reduce AHI prediction uncertainty from non-respiratory artifacts in EDR
Direct-from-ECG Desaturation: can ≥3% drop in blood oxygen be detected using ECG

Experiments were conducted that resulted in the development of an ECG-based alternative to the home sleep test (HST) for estimating an apnea-hypopnea index (AHI) and assessing OSA severity alongside quantification of AFib, Supraventricular, Junctional, Ventricular, Heart Block and Conduction defects with high sensitivity. Experiments were conducted to assess a novel ambulatory ECG-derived predictor of AHI in a prospective trial compared to an FDA-cleared WatchPAT HST.

39-subjects with prior diagnosis or positive screening for OSA on the STOP-Bang questionnaire were monitored using continuous ECG wearable for one night during a HST. AHI values (at oxygen desaturations of ≥3%) and total sleep time (TST) were derived from the HST report. ECG data matching the TST was processed to obtain ECG-derived Respiration (EDR) waveforms. Frequency and duration of obstructive events (OE), e.g., apnea & hypopneas, were computed using EDR. OEs from both LII, LIII EDR were refined through calculation of pre-, intra- and post-OE micro-arousal patterns using HRV analysis (3-15 sec duration) to filter out motion artifacts and respiratory-effort related arousals for enhancing AHI accuracy.

For 34 in-protocol participants, aged 24-70 with BMI 24.4-50.5 kg/m2, the AHI as output by HST ranged from 0.9 to 95.7. ECG-derived results yielded 91.2% predictive accuracy in diagnosing OSA compared to HST. Cohort's diagnosed co-morbidities included asthma, diabetes, obesity, hypertension, Heart Failure and hyperlipidemia. Positive predictive value was 100% in detecting sleep apnea in patients with either no OSA or severe OSA, aligning with the FDA-cleared HST. Three participants (7.7%) with mild or moderate OSA were predicted to have with 1 level higher OSA severity compared to HST. Rare VEs, SVEs, or SV rhythms implicated in OSA-to-AFib pathways were seen in study subjects diagnosed with OSA.

The results enable AHI direct-from-ECG with equivalent predictive value to AHI obtained using approved HST. This low-burden, low-cost HST alternative offers broad clinical utility for patients undergoing continuous ECG monitoring for arrhythmia, including SA outcomes. As a multi-night modality COR ECG provides time-distribution of obstructive events and AHI based on up to 7 sleep nights on night-by-night basis.

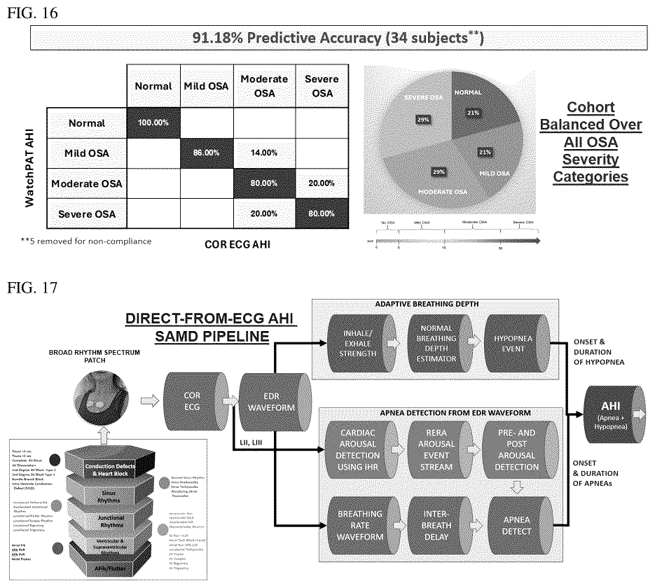

FIG. 16 summarizes results related to Example 4.

FIG. 17 summarizes results related to Example 4.

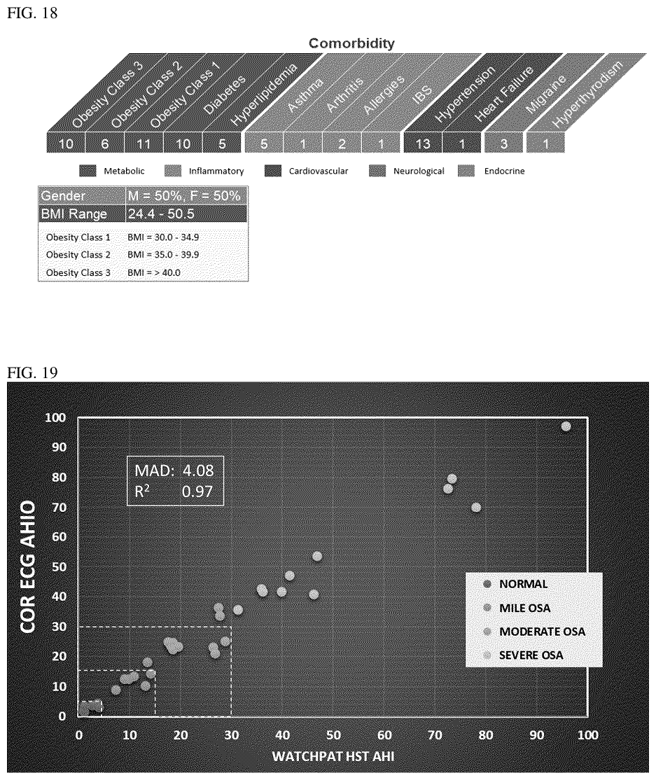

FIG. 18 summarizes results related to Example 4.

FIG. 19 summarizes results related to Example 4.

The following conclusions resulted from Example 4:
COR ECG-derived results achieved 91.2% predictive accuracy in diagnosing OSA compared to HST reference in a diverse test population;
Positive predictive value was 100% in detecting sleep apnea in patients with either no OSA or severe OSA, aligning with FDA-cleared HST results;
Rare ventricular events (VEs), supraventricular events (SVEs), or supraventricular rhythms implicated in OSA-to-atrial fibrillation (AFib) pathways were observed in study subjects diagnosed with OSA;
COR architecture based on Near-Field Einthoven's Triangle provides a robust inline mechanism to validate EDR-based obstructive events (OE) with ECG-based RERA marker;
Novel method for ECG-based hypopnea detection enabled.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes. Complete citations for the references cited within the application are provided within the following reference list.

1. A system for characterizing a subject's Ejection Fraction (EF) percentage status, comprising:
providing an electrocardiogram (ECG) device and a processor:
wherein the ECG device is wearable, wherein the ECG device has LII and LIII leads, wherein the ECG device is configured upon placement onto a subject to capture LII and LIII electrical signal waveform data from the subject via the LII and LIII leads over an extended period of time, wherein the ECG device is configured to wirelessly transmit LII and LIII electrical signal waveform data from the subject captured over an extended period of time to a processor, and
wherein the processor comprises software that, when executed, causes the processor to manually or automatically:
receive LII and LIII electrical signal waveform data captured over an extended period of time from a subject wirelessly transmitted from the ECG device; and
utilize the received LII and LIII electrical signal waveform data captured over an extended period of time to:
assess the subject's ejection fraction percentage status; and
provide a report to a user of the assessed ejection fraction percentage for the subject.

2. The system of claim 1, wherein the software, when executed, is configured to accomplish one or more of the following:
resample the LII and LIII electrical signal waveform data captured over an extended period of time to 250 Hz;
derive LI waveform with resampled LI and LII waveform data;
utilize the derived LI waveform and resampled LII and LIII waveform data to derive the following other bipolar frontal leads: aVF, aVR and aVL;
estimate a Wilson Central Terminal Reference potential with a spatial vector cardiography transform;
approximate a beat-by-beat maxima for pre-cordial leads V1 thru V6 with the spatial vector cardiography transform;
filter the derived LI waveform data, LII waveform data, LIII waveform data, aVF data, aVR data, aVL data, Wilson Central Terminal Reference potential, and V1 thru V6 data to remove motion (and other) artifacts;
calculate a spectral entropy for each heartbeat over the time period and remove all heartbeats above a pre-set threshold model;
compute PR, QT, ST interval for remaining stable heart-beats;
compute windowed HRV;
measure ventricular activation time (VAT) for derived lead aVR, wherein the VAT is measured from the onset of the QRS complex to the peak of the R wave;
measure P-wave terminal velocity, wherein the P-wave terminal velocity is measured by multiplying the amplitude of the derived V2 in millimeters (mm) and the time duration of the derived V2 negative deflection in milliseconds (ms);
measure Global QRS derivation, wherein measuring the Global QRS derivation comprises looking at the direction and magnitude of the QRS complexes in the derived LI waveform data, LII waveform data, LIII waveform data;
measure a vector of beat and rhythm burdens that includes quantified beat-type and rhythm with ST, PR, QT attributes;
synthesize the measured VAT, the measured P-wave terminal velocity, the measured Global QRS derivation, and the measured vector of beat and rhythm burdens into an Ejection Fraction (EF) biomarker value;
assess the subject's ejection fraction percentage status through comparing the subject's synthesized EF biomarker value with established EF biomarker value norms correlated with specific ejection fraction percentages; and
provide a report to a user of the assessed ejection fraction percentage for the subject.

3. The system of claim 2, wherein the software is configured to implement artificial intelligence (AI) based predictive analysis in assessing the subject's ejection fraction percentage status through comparing the subject's synthesized EF biomarker value with established EF biomarker value norms correlated with specific ejection fraction percentages.

4. The system of claim 1,
wherein the ECG has only two leads: LII and LIII, or
wherein the ECG has three leads including at least LII and LIII.

5. The system of claim 1, wherein the ECG device is a near-field continuous ECG recording device having three leads plus an on-body ground thereby providing two channels of ECG.

6. The system of claim 1, wherein the ECG device is placed onto the mid-center chest region of the subject.

7. The system of claim 1, wherein the ECG device includes LII and LIII frontal leads, and one V lead in any off-chest axis position.

8. The system of claim 1, wherein the ECG device includes LI, LII, and LIII leads in a 12-lead system and one V lead selected from V1, V2, V3, V4, V5, and V6.

9. The system of claim 1, wherein the ECG device is configured for placement onto any exterior portion of the subject's body.

10. The system of claim 1, wherein the ECG device is configured for placement onto the subject's chest, finger or wrist.

11. The system of claim 1, wherein the ECG device is any catheter system which provides at least 1 frontal lead and 1 precordial lead.

12. The system of claim 1, wherein the ECG device is any portable, tabletop, bedside telemetry ECG system which provides at least 1 frontal lead and 1 precordial lead.

13. The system of claim 1, wherein the ECG device is any episodic or continuous ECG system providing at least 10 seconds of continuous ECG data.

14. The system of claim 1, wherein the ECG device is any episodic or continuous ECG system providing at least 10 seconds of continuous ECG in conjunction with algorithm providing rhythm burden.

15. The system of claim 1, wherein the ECG device is any episodic or continuous ECG system providing at least 10 seconds of continuous ECG in conjunction with algorithm and Over-Read service providing rhythm burden.

16. The system of claim 1, wherein the ECG device is any episodic or continuous ECG system providing at least 10 seconds of continuous ECG in conjunction with Over-Read Service providing rhythm burden.

17. The system of claim 1, wherein the EF is left ventricular ejection fraction (LVEF).

18. The system of claim 1, wherein the subject is a human subject.

19. The system of claim 1, wherein the subject is a human subject experiencing or at risk of experiencing a cardiovascular event.

20. The system of claim 19, wherein the cardiovascular event is one or more cardiovascular events selected from heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

21. The system of claim 1, wherein the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

22. The system of claim 1, wherein the ECG device is configured to wirelessly transmit via Bluetooth, WI-FI, SD-card, and/or any type or kind of mobile data network.

23. The system of claim 1, wherein the ECG device includes the processor.

24. The system of claim 1, wherein the ECG device does not include the processor.

25. A method for characterizing a subject's Ejection Fraction (EF) percentage status, comprising:
providing a system recited in claim 1;
placing the ECG device onto the subject;
obtaining LII and LIII electrical signal waveform data captured over an extended period of time;
wirelessly transmitting the obtained LII and LIII electrical signal waveform data captured over an extended period of time to the processor via the ECG device;
executing the software to:
assess the subject's ejection fraction percentage status; and
provide a report to a user of the assessed ejection fraction percentage for the subject.

26. The method of claim 25, wherein executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value;
assessing the subject's ejection fraction percentage status through comparing the subject's synthesized EF biomarker value with established EF biomarker value norms correlated with specific ejection fraction percentages; and
providing a report to a user of the assessed ejection fraction percentage for the subject.

27. The method of claim 25,
wherein a subject's assessed ejection fraction percentage of equal to or greater than 52% for a male and equal to or greater than 54% for a female indicates a normal EF percentage;
wherein a subject's assessed ejection fraction percentage of 41% to 51% for a male and 41% to 53% for a female indicates a mildly abnormal EF percentage;
wherein a subject's assessed ejection fraction percentage of between 30% to 40% for a male or female indicates a moderately abnormal EF percentage;
wherein a subject's assessed ejection fraction percentage of less than 30% for a male or female indicates a severely abnormal EF percentage.

28. The method of claim 25, wherein the subject is a human subject.

29. The method of claim 25, wherein the subject is a human subject experiencing or at risk of experiencing a cardiovascular event.

30. The method of claim 29, wherein the cardiovascular event is one or more cardiovascular events selected from: heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

31. The method of claim 25, wherein the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 2 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

32. A method for measuring a subject's ECG Ventricular Activation Time (VAT), comprising:
providing a system recited in claim 1;
placing the ECG device onto the subject;
obtaining LII and LIII electrical signal waveform data captured over an extended period of time;
wirelessly transmitting the obtained LII and LIII electrical signal waveform data captured over an extended period of time to the processor via the ECG device;
executing the software to:
measure the subject's VAT; and
provide a report to a user of the measured VAT for the subject.

33. The method of claim 32, wherein executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value.

34. The method of claim 32, wherein the subject is a human subject.

35. The method of claim 32, wherein the subject is a human subject experiencing or at risk of experiencing a cardiovascular event.

36. The method of claim 32, wherein the cardiovascular event is one or more cardiovascular events selected from: heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

37. The method of claim 32, wherein the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 2 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

38. A method for measuring a subject's P-wave Terminal Velocity, comprising:
providing a system recited in claim 1;
placing the ECG device onto the subject;
obtaining LII and LIII electrical signal waveform data captured over an extended period of time;
wirelessly transmitting the obtained LII and LIII electrical signal waveform data captured over an extended period of time to the processor via the ECG device;
executing the software to:
measure the subject's P-wave Terminal Velocity; and
provide a report to a user of the measured P-wave Terminal Velocity for the subject.

39. The method of claim 38, wherein executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value.

40. The method of claim 38, wherein the subject is a human subject.

41. The method of claim 38, wherein the subject is a human subject experiencing or at risk of experiencing a cardiovascular event.

42. The method of claim 41, wherein the cardiovascular event is one or more cardiovascular events selected from: heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

43. The method of claim 38, wherein the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

44. A method of treating or preventing reduced ejection fraction (HFrEF) in a subject, comprising:
providing a system recited in claim 1;
assessing the ejection fraction percentage status for the subject as normal, mildly abnormal, moderately abnormal, or severely abnormal;
administering to the subject a therapeutic agent capable of treating or preventing heart failure with reduced ejection fraction if the subject's assessed ejection fraction percentage status is mildly abnormal, moderately abnormal, or severely abnormal.

45. The method of claim 44,
wherein a subject's assessed ejection fraction percentage of equal to or greater than 52% for a male and equal to or greater than 54% for a female indicates a normal EF percentage;
wherein a subject's assessed ejection fraction percentage of 41% to 51% for a male and 41% to 53% for a female indicates a mildly abnormal EF percentage;
wherein a subject's assessed ejection fraction percentage of between 30% to 40% for a male or female indicates a moderately abnormal EF percentage;
wherein a subject's assessed ejection fraction percentage of less than 30% for a male or female indicates a severely abnormal EF percentage.

46. The method of claim 44, wherein the therapeutic agent is selected from an ACE inhibitor, an ARB inhibitor, an ARB/neprilysin inhibitor, a beta blocker, an aldosterone antagonist, isosorbide dintrate/hydralazine, a diueretic, and ivabradine.

47. The method of claim 44, wherein the subject is a human subject experiencing or at risk of experiencing heart failure with HFrEF.

48. The method of claim 32, wherein the subject is a human subject experiencing or at risk of experiencing a cardiovascular event.

49. The method of claim 32, wherein the cardiovascular event is one or more cardiovascular events selected from: heart failure, congenital heart disease, heart attack, myocarditis, high blood pressure, low blood pressure, ATTR amyloidoisis, cardiotoxicity, ventricular arrhythmia, heart failure risk, cardiomyopathy, arrhythmias, impairment of cardiac pumping, etc.

50. A method for characterizing a subject's heart failure status, comprising:
providing a system recited in claim 1;
placing the ECG device onto the subject;
obtaining LII and LIII electrical signal waveform data captured over an extended period of time;
wirelessly transmitting the obtained LII and LIII electrical signal waveform data captured over an extended period of time to the processor via the ECG device;
executing the software to:
assess the subject's ejection fraction percentage status;
assess the subject's atrial enlargement status based on the measured P-wave Terminal Velocity; and
provide a report to a user of the assessed heart failure status for the subject.

51. The method of claim 50, wherein executing the software comprises one or more of the following:
resampling the LII and LIII electrical signal waveform data captured over an extended period of time to 250 Hz;
deriving LI waveform with resampled LI and LII waveform data;
utilizing the derived LI waveform and resampled LII and LIII waveform data to derive the following other bipolar frontal leads: aVF, aVR and aVL;
estimating a Wilson Central Terminal Reference potential with a spatial vector cardiography transform;
approximating a beat-by-beat maxima for pre-cordial leads V1 thru V6 with the spatial vector cardiography transform;
filtering the derived LI waveform data, LII waveform data, LIII waveform data, aVF data, aVR data, aVL data, Wilson Central Terminal Reference potential, and V1 thru V6 data to remove motion (and other) artifacts;
calculating a spectral entropy for each heartbeat over the time period and remove all heartbeats above a pre-set threshold model;
computing PR, QT, ST interval for remaining stable heart-beats;
computing windowed HRV;
measuring ventricular activation time (VAT) for derived lead aVR, wherein the VAT is measured from the onset of the QRS complex to the peak of the R wave;
measuring P-wave terminal velocity, wherein the P-wave terminal velocity is measured by multiplying the amplitude of the derived V2 in millimeters (mm) and the time duration of the derived V2 negative deflection in milliseconds (ms);
measuring Global QRS derivation, wherein measuring the Global QRS derivation comprises looking at the direction and magnitude of the QRS complexes in the derived LI waveform data, LII waveform data, LIII waveform data;
measuring a vector of beat and rhythm burdens that includes quantified beat-type and rhythm with ST, PR, QT attributes;
synthesizing the measured VAT, the measured P-wave terminal velocity, the measured Global QRS derivation, and the measured vector of beat and rhythm burdens into an Ejection Fraction (EF) biomarker value;
assessing the subject's ejection fraction percentage status through comparing the subject's synthesized EF biomarker value with established EF biomarker value norms correlated with specific ejection fraction percentages; and
providing a report to a user of the assessed ejection fraction percentage for the subject.

52. The method of claim 50, wherein the subject is a human subject.

53. The method of claim 50, wherein the subject is a human subject experiencing or at risk of experiencing heart failure.

54. The method of claim 50, wherein the subject is a human subject experiencing or at risk of experiencing HFrEF or HFpEF.

55. The method of claim 50, wherein the extended period of time is:
between a 0.00001-second period of time and less than or equal to a 6-month period of time, or
between a 1 second period of time and less than or equal to a one-month period of time, or
between a 1-second period of time and less than or equal to a two-week period of time, or
between a 1-second period of time and less than or equal to a one-week period of time, or
between a 1-second period of time and less than or equal to a 72-hour period of time, or
between a 1-second period of time and less than or equal to a 48-hour period of time, or
between a 1-second period of time and less than or equal to a 24-hour period of time, or
between a 1-second period of time and less than or equal to a 12-hour period of time, or
between a 1-second period of time and less than or equal to a 6-hour period of time, or
between a 1-second period of time and less than or equal to a 3-hour period of time, or
between a 1-second period of time and less than or equal to a 1-hour period of time, or
between a 1-second period of time and less than or equal to a 45-minute period of time, or
between a 1-second period of time and less than or equal to a 30-minute period of time, or
between a 1-second period of time and less than or equal to a 15 minute period of time, or
between a 1-second period of time and less than or equal to a 10 minute period of time, or
between a 1-second period of time and less than or equal to a 5 minute period of time, or
between a 1-second period of time and less than or equal to a 4 minute period of time, or
between a 1-second period of time and less than or equal to a 3 minute period of time, or
between a 1-second period of time and less than or equal to a 2 minute period of time, or
between a 1-second period of time and less than or equal to a 1 minute period of time, or
between a 1-second period of time and less than or equal to a 45-second period of time, or
between a 1-second period of time and less than or equal to a 30-second period of time, or
between a 1-second period of time and less than or equal to a 15-second period of time, or
between a 1-second period of time and less than or equal to a 10-second period of time, or
between a 1-second period of time and less than or equal to a 9-second period of time, or
between a 1-second period of time and less than or equal to a 5-second period of time, or
between a 1-second period of time and less than or equal to a 3-second period of time, or
between a 1-second period of time and less than or equal to a 2-second period of time.

56. A method for estimating an apnea-hypopnea index (AHI) and assessing OSA severity alongside quantification of AFib, Supraventricular, Junctional, Ventricular, Heart Block and Conduction defects with high sensitivity, comprising providing the system recited in claim 1, and implementing the technique recited in Example 4.
